(19) United States
(12) Patent Application Publication
(10) Pub. No.: US 2014/0324129 A1
(43) Pub. Date: Oct. 30, 2014

(54) SYSTEMS AND METHODS FOR TEMPORARY, INCOMPLETE, BI-DIRECTIONAL, ADJUSTABLE ELECTRICAL NERVE BLOCK

(71) Applicant: Case Western Reserve University, Cleveland, OH (US)

(72) Inventors: Manfred Franke, Cleveland, OH (US); Ashritha Epur, Cleveland, OH (US); Benjamin Kaufmann, Potomac, MD (US)

(73) Assignee: Case Western Reserve University, Cleveland, OH (US)

(21) Appl. No.: 14/266,027

(22) Filed: Apr. 30, 2014

Related U.S. Application Data
(60) Provisional application No. 61/817,629, filed on Apr. 30, 2013.

(51) Int. Cl. A61N 1/36; A61N 1/05

(52) U.S. Cl. CPC A61N 1/3606; A61N 1/0551; A61N 1/0556; A61N 1/0553
USPC 607/62; 607/116; 607/118; 607/117

(57) ABSTRACT

One aspect of the present disclosure relates to a system that can provide an incomplete nerve block to a patient. In some instances, the incomplete nerve block can be bi-directional. In other instances, the incomplete nerve block can be adjustable. The system can include a waveform generator that can provide temporary electrical nerve conduction block to a nerve using an electrode. The electrode can include at least one contact. The temporary electrical nerve conduction block can block conduction in less than 100% of the fibers within the nerve located in close proximity to or being surrounded by the electrode. The temporary electrical nerve conduction block does not cause intentional damage to neural tissue as mode of action to achieve the incomplete nerve block. A complete recovery of nerve conduction can be expected post application of the incomplete nerve block.

### RELATED APPLICATIONS

This application claims the benefit of U.S. Provisional Application No. 61/817,629, filed Apr. 30, 2013, entitled “DIRECTLY-MODULATED NERVE CONDUCTION USING PATIENT-CONTROLLED KHFAC AND/OR DC BLOCK AND/OR COMBINED KHFAC+DC BLOCK” the entirety of which is hereby incorporated by reference for all purposes.

### TECHNICAL FIELD

The present disclosure relates generally to electrical nerve block and, more specifically, to systems and methods that can provide a temporary, incomplete, bi-directional, adjustable electrical nerve block to at least a portion of a nerve.

### BACKGROUND

Muscle spasticity is a significant co-morbidity of neurological disorders, such as stroke, brain injury, spinal cord injury (SCI), cerebral palsy (CP), and multiple sclerosis (MS). Spasticity presents as unwanted, uncontrolled, and/or uncoordinated muscle contractions resulting in stiffness around joints and loss of coordination. Many patients suffering from spasticity can feel different levels of spasticity on different days and even at different times during a single day (e.g., morning vs. evening).

Conventionally, spasticity is managed by a hierarchy of increasingly invasive methods, including: physiotherapy, bracing, oral medications, injections, chemical neurolysis, intrathecal medications, and surgery. In some cases, a neural prosthesis can deliver an electrical signal to the nerve that is strong enough to stop conduction in substantially all fibers within the nerve (“complete nerve block”). The electrical nerve block can substantially eliminate spasticity, but can also eliminate conduction in other fibers in the nerve not contributing to the spasticity.

### SUMMARY

The present disclosure relates generally to electrical nerve block and, more specifically, to systems and methods that can provide a temporary, incomplete, bi-directional, adjustable electrical nerve block to at least a portion of a nerve. In other words, the incomplete electrical nerve block can block the conduction in at least a portion of the nerve without causing intentional damage of neural tissue as mode of action to achieve the incomplete nerve block during application of the electrical nerve conduction block. In some instances, the temporary, incomplete, bi-directional electrical nerve block can be adjustable (e.g., controlled by a patient, a medical professional, or autonomously based on a condition associated with the patient). In other instances, the temporary, incomplete, bi-directional electrical nerve block can be applied to a portion of the fibers within the nerve while a stimulus pulse can be applied to another portion of the fibers within the nerve. In some instances, a grading scale for the intensity of the partial block can be determined using waveform parameters that provide 0% and 100% block, as well as various percentages of block in between. In other words, complete nerve block can be used as a tool to determine the boundary conditions for optimal block parameters.

In one aspect, the present disclosure can include a system that provides a bi-directional, incomplete nerve block to a patient. The system can include a waveform generator configured to modulate a parameter of an electrical waveform so that the electrical waveform is configured to temporarily block conduction in less than 100% of the fibers within a portion of a nerve in close proximity to the electrode. The system can also include an electrode, electrically coupled to the waveform generator, located in proximity to a nerve and configured to deliver the electrical conduction block waveform to the nerve via at least one contact.

In another aspect, the present disclosure can include a method for providing a bi-directional, incomplete nerve block to a patient. The method can include the step of providing an adjustable (on demand and instantaneous) temporary electrical nerve conduction block to a nerve using an electrode. The method can also include the step of blocking conduction in less than 100% of the fibers within the nerve located in close proximity to or being surrounded by the electrode without causing intentional damage of neural tissue as mode of action to achieve the incomplete nerve block during application of the electrical nerve conduction block. A complete recovery of nerve conduction can be expected post application of the incomplete nerve block.

In a further aspect, the present disclosure can include a neural prosthesis. The neural prosthesis can include an external control unit configured to receive an input from a patient. The input modulates a parameter of an electrical waveform. The external control unit can include a user interface, a non-transitory memory, and a processor. The neural prosthesis can also include a waveform generator configured to adjust the electrical waveform based on the input and provide the adjusted electrical waveform to a nerve via an electrode. The electrical waveform can be configured to provide a temporary, incomplete, bi-directional nerve block to the nerve at the electrode site.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a schematic block diagram showing a system that can provide a incomplete, bi-directional electrical nerve block to a patient in accordance with an aspect of the present disclosure;

FIG. 2 is a schematic block diagram showing a system that can provide an adjustable, incomplete, bi-directional electrical nerve block to a patient in accordance with an aspect of the present disclosure;

FIGS. 3-5 are schematic block diagrams showing example configurations of a controller that can be part of the system shown in FIG. 2;

FIG. 6 is a schematic block diagram showing example configurations of the system shown in FIG. 2;

FIG. 7 is a process flow diagram illustrating a method for providing a incomplete, bi-directional electrical nerve block to a patient in accordance with another aspect of the present disclosure; and

FIG. 8 is a process flow diagram illustrating a method for altering a parameter of the incomplete, bi-directional electrical nerve block of the method shown in FIG. 7.

### DETAILED DESCRIPTION

### I. Definitions

In the context of the present disclosure, the singular forms “a,” “an” and “the” can also include the plural forms, unless the context clearly indicates otherwise. The terms “comprises” and/or “comprising,” as used herein, can specify the presence of stated features, steps, operations, elements, and/or components, but do not preclude the presence or addition of one or more other features, steps, operations, elements, components, and/or groups. As used herein, the term “and/or” can include any and all combinations of one or more of the associated listed items. Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “nerve” can refer to a “peripheral nerve.” Generally, a peripheral nerve can refer to a nerve in a patient's body other than brain and spinal cord. A peripheral nerve can include a bundle of fibers (including motor and sensory fibers) that can connect the brain and spinal cord to the rest of the patient's body. For example, a peripheral nerve can control the functions of sensation, movement, and motor coordination. In some instances, the peripheral nerve can conduct information bi-directionally (e.g., providing both motor control and sensory feedback).

As used herein, the term “fiber” can refer to an appendage of a neuron that can transmit impulses away from the soma of the neuron to the synaptic terminal of the neuron. In some instances, the fiber can conduct action potentials in both directions (e.g., to and from the soma). The terms “fiber” and “axon” can be used interchangeably herein. Additionally, the terms “conduct information” and “action potential” can be used interchangeably herein.

As used herein, the term “nerve block” can refer to an arrest and/or failure of the transmission (also referred to as passage or conduction) of action potentials at some point along a nerve. A nerve block can provide a complete nerve block or a partial nerve block that is reversible and not destructive. Examples of types of nerve block can include nerve conduction block, synaptic-junction-depletion block, uni-directional block (blocks conduction in one direction), bi-directional block (blocks conduction in two directions). Although “nerve conduction block” is a subset of “nerve block,” the term “nerve conduction block,” as used herein, can encompass all types of reversible, non-destructive nerve block.

As used herein, the term “complete nerve block” can refer to a nerve block that can inhibit the transmission of action potentials on substantially all (e.g., 100%) of the fibers of the nerve. With complete nerve block, the sensory fibers, motor fibers, sympathetic fibers, and parasympathetic fibers in the nerve are assumed to be all blocked. Both the large fibers of the nerve and the small fibers of the nerve are assumed to be blocked. Additionally, both afferent and efferent fibers are also blocked.

As used herein, the term “incomplete nerve block” can refer to a nerve block that inhibits the transmission of action potentials on less than all (e.g., less than 100%) of the fibers in the nerve, or less than 100% of the fibers of the nerve with similar characteristics (e.g., fiber size or diameter, afferent or efferent, motor or sensory, sympathetic or parasympathetic, etc.). The incomplete nerve block can be temporary and bi-directional. In some instances, the incomplete nerve block can block the transmission of action potentials in at least 10% of the fibers within the nerve and not more than 90% of the fibers within the nerve. In some instances, large fibers can be blocked before small fibers, or vice versa. In other instances, afferent fibers and efferent fibers can be blocked at different percentages. In further instances, a majority of alpha-motor fibers of the nerve can be blocked, while a minimal number of sensory fibers within the nerve can be blocked, or vice versa. In further instances, a majority of sympathetic fibers of the nerve can be blocked, while a minimal number of parasympathetic fibers within the nerve can be blocked, or vice versa. The terms “incomplete nerve block” and “partial nerve block” can be used interchangeably herein.

As used herein, the term “graded nerve block” can refer to an adjustable incomplete nerve block. In some instances, the graded nerve block can be adjusted between different percentages of fibers to block within the nerve. For example, the adjustment can range from zero fibers blocked to less than 100% of fibers blocked (e.g., 0% block and 100% block can be used as boundary conditions to set the end points for the graded block). In other instances, the graded nerve block can refer to a full nerve block that is obtained in one portion of the nerve (e.g., a bundle including several, but not all, fibers of the nerve), while a reduced block or not block is applied to a second portion of the nerve. For example, a block can be achieved on the left side of the nerve, but not on the right side of the nerve. In some instances, the graded nerve block can be applied differently between different contacts of the same electrode.

As used herein, the terms “nerve block modality” or “nerve conduction block signal” can refer to a signal that can provide complete nerve block and/or incomplete nerve block, but not destructive block. Examples of nerve block modalities include: an electrical waveform, a light signal, an ultrasound signal, and a heating, or a cooling signal.

As used herein, the term “electrical waveform” can refer to an electrical signal that can be applied to the nerve with an electrode to achieve the incomplete nerve block. In some instances, the electrical waveform can be a mathematical description of a change in voltage or current over time. The electrical waveform can be defined by one or more parameters. For example, the parameters can be one or more of frequency, amplitude, polarity, a pause in the waveform, waveform shape (triangle, rectangle, sinus, etc.), and period. Examples of electrical waveforms that can provide the incomplete block include: high frequency electric alternating current (HFAC) waveform, a kilohertz HFAC (KHFAC), a charge-balanced direct current (CBDC) waveform, or a multi-phased direct current (MPDC) waveform. Although the term “electrical waveform” has a specific meaning, it is used herein encompassing all of the “nerve block modalities” unless expressed otherwise. The terms “electrical waveform” and “electrical signal” can be used interchangeably herein. Additionally, the terms “modification,” “alteration,” “adjustment,” as well as any term conveying similar meaning, can be used interchangeably herein to describe a change to the electrical signal.

As used herein, the term “electrode” can refer to a device that provides an attachment for one or more contacts. The one or more contacts can be made of an interface material providing the conversion of current flow via electrons in a metal (wire/lead) to ionic means (in an electrolyte, such as interstitial fluid). In some instances, the electrode can aid in shaping the electric field generated by the contacts.

As used herein, the term “waveform generator” can refer to a device that can provide the electrical signal to the electrode. For example, the waveform generator can be connected to the electrode via one or more leads. In some instances, the waveform generator can be implanted within a patient's body. In other instances, the waveform generator can be external to the patient's body.

As used herein, the term “controller” can refer to a device that can be used to modify or control the output electrical signal of the waveform generator. In some instances, the controller can receive an input (e.g., from a patient, a medical professional, and/or a sensor) adjusting one or more parameters of the electrical signal. In other instances, the controller can be programmable (e.g., by a medical professional) to correspond to the patient.

As used herein, the term “sensor” can refer to a device that can detect and/or measure one or more parameters related to the patient and/or the external environment. Examples of parameters that can be detected by the sensor can include an activity level of the patient determined from an electroencephalogram (EEG), an electromyogram (EMG), an electrocardiogram (ECG), a breathing change, a pO₂ change, a pCO₂ change, an accelerometer measurement, a blood sugar change, a temperature change of the patient, and/or a specific time during the day/night cycle.

As used herein, the term “temporary” can refer to a period of time with a finite end point. For example, a temporary incomplete block can be applied for a finite period of time without causing intentional damage of neural tissue as mode of action to achieve the incomplete nerve block during application of the electrical nerve conduction block.

As used herein, the term “neural prosthesis” or “neuralprosthetic” can refer to one or more devices that can substitute for a neurological function (e.g., motor function, sensory function, cognitive function, etc.) that has been damaged (e.g., as a result of a neurological disorder, injury, and/or accident). For example, a neural prosthesis can include a stimulation device that restores neurological function and/or a blocking device that blocks nerve conduction. In some instances, the neural prosthesis can provide the incomplete nerve block.

As used herein, the term “neurological disorder” can refer to a condition of disease characterized at least in part by abnormal (or unwanted) conduction in one or more nerves. In some instances, the abnormal conduction can relate to pain and/or spasticity. In other instances, the abnormal conduction can relate to sympathetic or parasympathetic over-activity. In further instances, the abnormal conduction can relate to pathological conditions that are not necessarily of direct neural origin but can be provided symptom relief, treatment, or cure using partial activation or partial block of certain nerve fibers. Examples of neurological disorders can include stroke, brain injury, spinal cord injury (SCI), cerebral palsy (CP), and multiple sclerosis (MS), obesity, elevated blood pressure or heart rate, pathologically elevated sympathetic or parasympathetic tone on one or more peripheral nerves.

As used herein, the term “medical professional” can refer to can refer to any person involved in medical care of a patient including, but not limited to, physicians, medical students, nurse practitioners, nurses, and technicians.

As used herein, the term “patient” can refer to any warm-blooded organism including, but not limited to, a human being, a pig, a rat, a mouse, a dog, a cat, a goat, a sheep, a horse, a monkey, an ape, a rabbit, a cow, etc. The terms “patient” and “subject” can be used interchangeably herein.

### II. Overview

The present disclosure relates generally to electrical nerve block and, more specifically, to systems and methods that can provide a temporary, incomplete, bi-directional, adjustable electrical nerve block to at least a portion of a nerve. In other words, the incomplete electrical nerve block can block the conduction in at least a portion of the nerve without intentionally damaging the nerve during application of the electrical nerve conduction block. In some instances, the temporary incomplete electrical nerve block can be adjustable (e.g., controlled by a patient, a medical professional, or autonomously based on a condition of the patient). In other instances, the temporary, incomplete electrical nerve block can be applied to a portion of the fibers within the nerve while a stimulus pulse can be applied to another portion of the fibers within the nerve.

In some instances, the incomplete electrical nerve block can be used for spasticity control (e.g., providing variable levels of block for different patient activity levels). In other instances, the incomplete electrical nerve block can block fibers contributing to an unwanted action, while allowing conduction through fibers that do not contribute to the unwanted action (e.g., blocking motor fibers, while allowing conduction through sensory fibers). In still other instances, the incomplete nerve block can drive smaller fibers within a nerve, while blocking larger fibers within the nerve (e.g., for select muscle drive of slow-fatiguing muscle fibers). In further instances, the incomplete nerve block can permit selection between sympathetic and parasympathetic fibers (e.g., within nerves spanning between organs and the central nervous system). In still further instances, the incomplete electrical nerve block can be used as a filter combined with full nerve drive to achieve small fiber activation without activating the large fibers within the nerve. In yet other instances, the incomplete nerve block can be applied by one contact of an electrode (e.g., a nerve re-shaping electrode, such as a flat interface nerve electrode) and no block or a driving electrical stimulation can be applied by a second contact of the electrode (e.g., for select muscle drive of slow-fatiguing muscle fibers or for select sensory fiber drive to modulate the sympathetic vs. parasympathetic balance of a patient).

### III. Systems

One aspect of the present disclosure can include a system that can that can provide a temporary, incomplete, bi-directional nerve block to a patient. The incomplete nerve block can provide a temporary block to at least a portion of fibers within a nerve (e.g., less than 100%). Advantageously, the incomplete electrical nerve block can block the conduction in at least a portion of the nerve without intentionally damaging the nerve during application of the electrical nerve conduction block. The incomplete nerve block can be adjustable based on an input by a patient, a medical professional, or data from a sensor, instantaneously and on demand. In other words, the incomplete nerve block can be an intended, graded nerve block (providing an effect rather than a side effect).

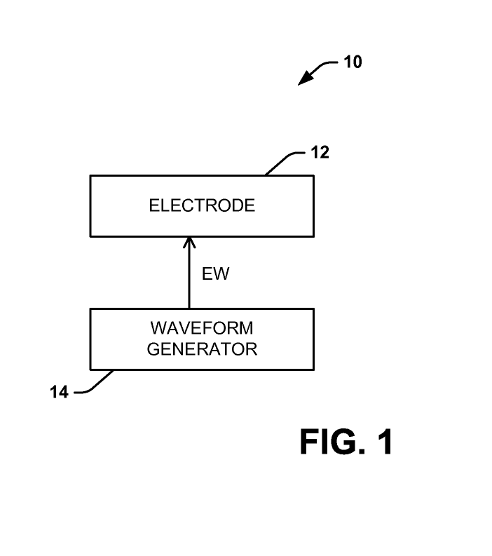

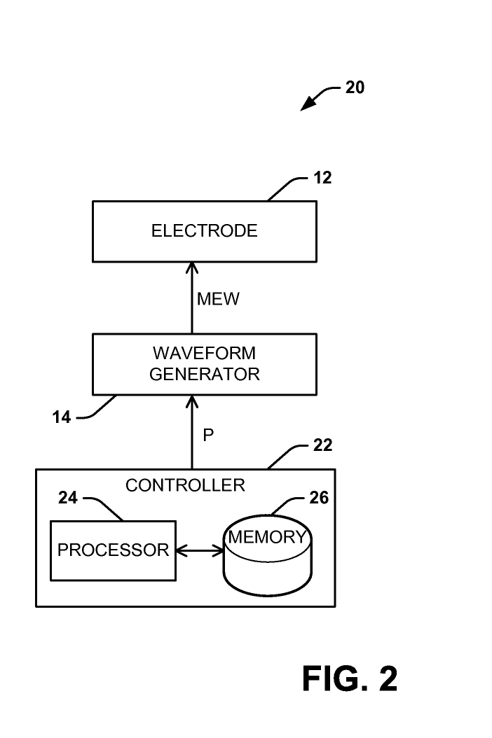

FIG. 1 illustrates an example of a system 10 that can that can provide an incomplete, adjustable (on demand and instantaneous) nerve block to a patient, according to an aspect of the present disclosure. FIG. 1, as well as associated FIGS. 2-6, are schematically illustrated as block diagrams with the different blocks representing different components. The functions of one or more of the components (e.g., the controller 22) can be implemented by computer program instructions. These computer program instructions can be provided to a processor of a general purpose computer, special purpose computer, and/or other programmable data processing apparatus to produce a machine, such that the instructions, which execute via the processor of the computer and/or other programmable data processing apparatus, create a mechanism for implementing the functions of the components specified in the block diagrams.

These computer program instructions can also be stored in a non-transitory computer-readable memory that can direct a computer or other programmable data processing apparatus to function in a particular manner, such that the instructions stored in the non-transitory computer-readable memory produce an article of manufacture including instructions, which implement the function specified in the block diagrams and associated description.

The computer program instructions can also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions of the components specified in the block diagrams and the associated description.

Accordingly, the controller 22 described herein can be embodied at least in part in hardware and/or in software (including firmware, resident software, micro-code, etc.). Furthermore, aspects of the controller 22 can take the form of a computer program product on a computer-usable or computer-readable storage medium having computer-usable or computer-readable program code embodied in the medium for use by or in connection with an instruction execution system. A computer-usable or computer-readable medium can be any non-transitory medium that is not a transitory signal and can contain or store the program for use by or in connection with the instruction or execution of a system, apparatus, or device. The computer-usable or computer-readable medium can be, for example but not limited to, an electronic, magnetic, optical, electromagnetic, infrared, or semiconductor system, apparatus or device. More specific examples (a non-exhaustive list) of the computer-readable medium can include the following: a portable computer diskette; a random access memory; a read-only memory; an erasable programmable read-only memory (or Flash memory); and a portable compact disc read-only memory.

As shown in FIG. 1, one aspect of the present disclosure can include a system 10 configured to provide an incomplete, bi-directional nerve block to a portion of a nerve in a patient's body. In some instances, the incomplete, bi-directional nerve block can block the conduction in less than 100% of the fibers in the nerve. In other instances, the incomplete, bi-directional nerve block can block the conduction in from at least 10% to 90% of the fibers in the nerve. As noted above, the incomplete, bi-directional electrical nerve block advantageously can block the conduction within the portion of the fibers in the nerve without causing intentional damage to neural tissue as a mode of action to achieve the incomplete nerve block during application of the incomplete nerve block.

The system 10 can include components including at least an electrode 12 and a waveform generator 14. The waveform generator 14 can deliver an electrical signal (EW) to the electrode 12 for application of an incomplete, bidirectional nerve block to a nerve. The electrode 12 and the waveform generator 14 can be communicatively coupled. In some instances, the electrode 12 and the waveform generator 14 can be communicatively coupled via one or more wired leads. In other instances, the electrode 12 and the waveform generator 14 can be communicatively coupled via an indirect coupling (e.g., capacitive coupling and/or inductive coupling).

The electrode 12 can include one or more contacts to deliver the incomplete, bidirectional nerve block to a nerve. In some instances, the incomplete, bidirectional nerve block can be delivered to the nerve by the electrode 12 by applying the electrical signal (EW) to an area of the nerve in proximity to or surrounded by the electrode and/or the electrode contact. Examples of electrodes that can be used as electrode 12 include a nerve shaping electrode, an electrode array, a spiral electrode, a cuff electrode, a Huntington style electrode, a co-linear placed spinal cord stimulation (SCS) or deep brain stimulation (DBS) electrode, a disk electrode, an intra-muscular electrode, or an intra-fascicular electrode.

In some instances, the electrode 12 can include at least a first contact and a second contact. The first contact can deliver the electrical signal to a portion of the nerve to incompletely block conduction within a portion of the nerve. In some instances, the second contact can deliver no electrical signal to the nerve. In other instances, the second contact can deliver a second electrical signal to a second portion of the nerve to generate action potentials in the fibers within the second portion of the nerve. In some instances the second portion of the nerve can overlap the first portion of the nerve. In other instances, the second portion of the nerve can be separate from the first portion of the nerve (e.g., does not overlap).

The waveform generator 14 can provide an electrical signal to the electrode 12 for application to the nerve to facilitate the incomplete, bi-directional block. The electrode 12 can include one or more contacts that can apply the electrical signal to an area of the nerve in proximity to or surrounded by the electrode. The block can be achieved at the area of the nerve in response to the electrical signal. In some instances, the incomplete, bi-directional nerve block can be temporary, with a finite duration. For example, the duration of the block can be defined by one or more parameters related to the electrical signal.

The waveform generator can deliver the electrical signal (EW) to the electrode 12 for application to the nerve. In some instances, the waveform generator 14 can be located external to the patient's body. In other instances, the waveform generator 14 can be located internal to the patient's body. In still other instances, one part of the waveform generator 14 can be located internal to the patient's body, while the other part of the waveform generator 14 can be located external to the patient's body. For example, the waveform generator 14 can include at least an over-voltage controller (such as a diode) in addition to a coiled wire (e.g., a secondary coil of a transformer) that can bridge the patient's skin. However, the waveform generator 14 can be of higher levels of complexity beyond a coiled wire.

In some instances, the waveform generator 14 can be communicatively coupled to a controller 22, as shown in system 20 of FIG. 2. In some instances, system 20 can be a modification to an existing neural prosthesis (that is capable of providing a complete nerve block) to modulate the electrical output signal of the neural prosthesis in such a way so that the complete nerve block becomes an incomplete nerve block that affects less than 100% of the fibers within the nerve. In other instances, system 20 can be a stand-alone neural prosthesis capable of delivering a graded nerve block (e.g., from 0% to less than 100% of the fibers in the nerve blocked). For example, values corresponding to the 0% nerve block and the 100% nerve block can be used as end points that determine a range for usefulness of the stimulation (e.g., these values can be used to define a safety boundary and/or an efficiency boundary).

The controller 22 can be external to the patient's body and configured to receive an input altering one or more parameters of the electrical signal. In some instances, the parameter can be frequency. Altering the frequency can change the intensity of the block without altering any other parameters of the electrical signal. In other instances, the parameter can include both frequency and amplitude, which can change the intensity of the block without altering any other parameters of the electrical signal.

The controller 22 can deliver the modulated parameter (P) to the waveform generator 14, which can modulate the electrical signal according to the input. The waveform generator 14 can deliver the modulated electrical signal (MEW) to the electrode 12 for application of the incomplete, bidirectional nerve block to the nerve.

In some instances, the input can be received from the patient, received from a medical professional, and/or based on a status of the patient's body based on data received from a sensor. In some instances, the change of the one or more parameters can relate to the intensity of the incomplete, bi-directional nerve block. For example, the intensity of the block can be changed between values from 0% to less than 100% so that the incomplete, bidirectional nerve block can be a graded nerve block.

The controller 22 can send an indication of the altered parameter (P) to the waveform generator. In some instances, the controller 22 and the waveform generator 14 can be communicatively coupled via a wired connection. In other instances, the controller 22 and the waveform generator 14 can be indirectly coupled via a wireless connection (e.g., the controller can have a transmitter and the waveform generator can have a receiver).

In some instances, the controller 22 can include a magnet. The input can be received by swiping the magnet over the location of the waveform generator 14 (that can be at least partially implanted within the body). The number and intensity of the swipe can correspond to a switch of intensity of the block. For example, one swipe can correspond to a 20% intensity of the block; two swipes can correspond to a 50% intensity of the block; and three swipes can correspond to an 85% intensity of the block. For example, a patient could wear the controller 22 as a small wrist band with an embedded magnet.

In other instances, the controller 22 can be at least a portion of a computerized device, such as a smart phone or a tablet computing device. The controller 22 can include a non-transitory memory 26 and a processor 24. For example, the non-transitory memory 26 can store instructions associated with the operation of the controller 22 (e.g., associated with adjusting the parameter of the electrical signal) can be stored in the non-transitory memory 26. The processor 24 can facilitate execution of the instructions stored in the non-transitory memory 26. For example, the instructions can be part of a software application that can communicate with the waveform generator 14.

The controller 22 can provide a patient and/or medical personnel with the ability to interface with the waveform generator 14. The patient and/or medical personnel can modulate a parameter at the controller 22, which can communicate the modulated parameter (P) to the waveform generator 14. The waveform generator 14 can modulate the electric signal (MEW) according to the modulated parameter to modulate the level of block. Additionally or alternatively, the electric signal can also be modulated in response to information related to the status of the patient (e.g., received from a sensor). Examples of parameters that can be detected by the sensor can include an activity level of the patient determined from an electroencephalogram (EEG), an electromyogram (EMG), an electrocardiogram (ECG), a breathing change, a pO₂ change, a pCO₂ change, an accelerometer measurement, a blood sugar change, a temperature change of the patient, and/or a specific time during the day/night cycle. For example, a patient can receive a full block or a higher grade (intensity) block at night so the patient can achieve sleep, and a lower intensity block during the day so the patient can have more control or strength (e.g., for walking).

In some instances, the controller 22 can provide power to the waveform generator 14 and any other components internal to the patient's body. Accordingly, the waveform generator 14 need not include any independent power generation components.

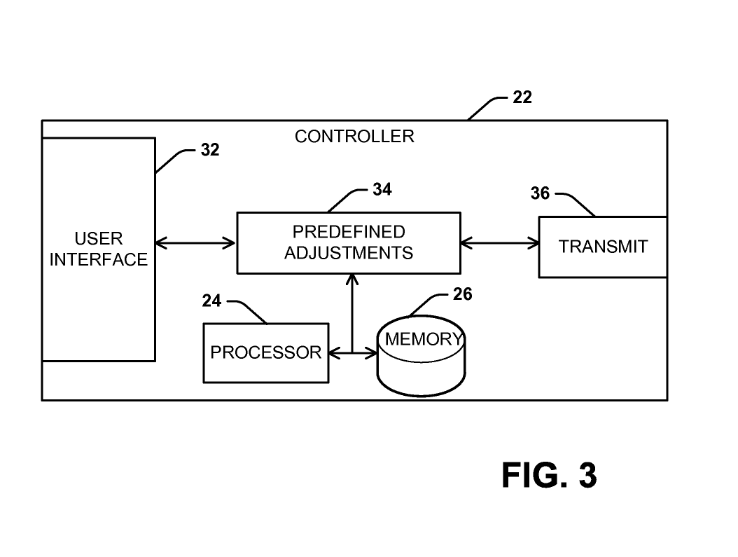

Example configurations of the controller 22 with different instructions stored in the non-transitory memory 26 are shown in FIGS. 3-5. These example configurations are not meant to be exclusive. Additionally, each of FIGS. 3-5 can be included within the same controller or different controllers.

FIG. 3 illustrates the controller 22 allowing the patient and/or medical professional to enter an input corresponding to a selection of a predefined parameter alteration. The controller 22 can include a user interface 32 that can display the plurality of predefined parameter alterations and receive an input selecting one of the plurality of predefined parameter alterations. The predefined parameter alterations can be stored in the non-transitory memory 26 and accessed via a predefined alterations 34 component. The selected predefined parameter alteration can be provided to the waveform generator 14 via the transmission component 36. The transmission component 36 can be configured for wired transmission of the selected predefined parameter and/or wireless transmission of the selected predefined parameter.

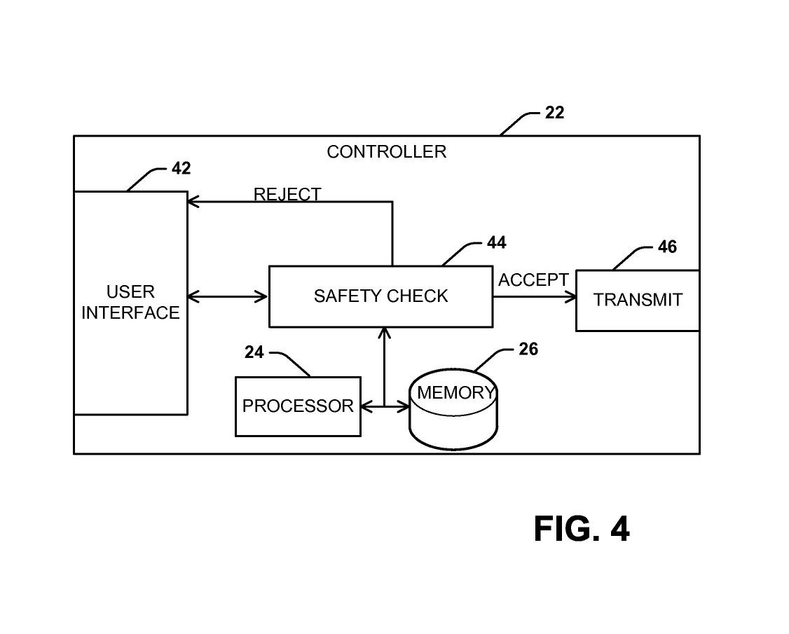

FIG. 4 illustrates the controller 22 allowing the patient to enter alterations to a parameter freely without choosing between predefined parameters. The controller 22 can include a user interface 42 that can receive the alteration to the parameter. The alteration can be verified by a safety check component 44. For example the safety check component can compare the parameter modified according to the alteration to a predefined safety boundary (to determine whether the modified electrical waveform will have the potential to cause injury to the nerve) and/or a predefined efficiency boundary (to determine whether the modified electrical waveform will be effective at providing the desired level of incomplete block). The predefined safety boundary and/or the predefined efficiency boundary can be stored in the non-transitory memory 26. When the safety check component 44 determines that the parameter modified according to the alteration is beyond the safety boundary and/or the efficiency boundary, the safety check component rejects the alteration. When the safety check component 44 determines that the alteration keeps the altered parameter within the safety boundary and the efficiency boundary, the altered parameter can be sent (by a transmission component 46) to the waveform generator 14 via a wired transmission or a wireless transmission.

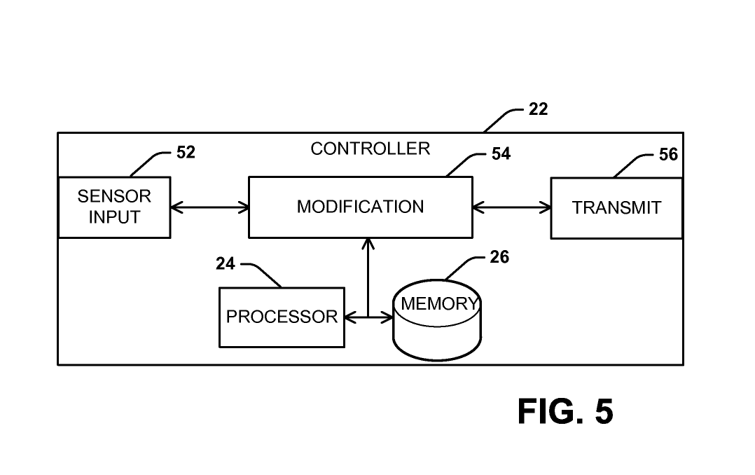

FIG. 5 illustrates the controller 22 that can receive an input 52 from a sensor and alter the parameter based on the sensor input. In some instances, the sensor input can correspond to a status of a patient and/or a status of an environment related to the patient. Examples of status parameters that can be detected by the sensor can include a patient physiological parameter, a patient activity, a patient position, a patient acceleration, a time of day, and a relative position of the patient's body. In response to the input 52, the parameter of the electrical signal can be changed (e.g., by a modification component 54), and the changed parameter of the electrical signal can be sent to the waveform generator 14 (by a transmission component 56) to the waveform generator via a wired transmission or a wireless transmission.

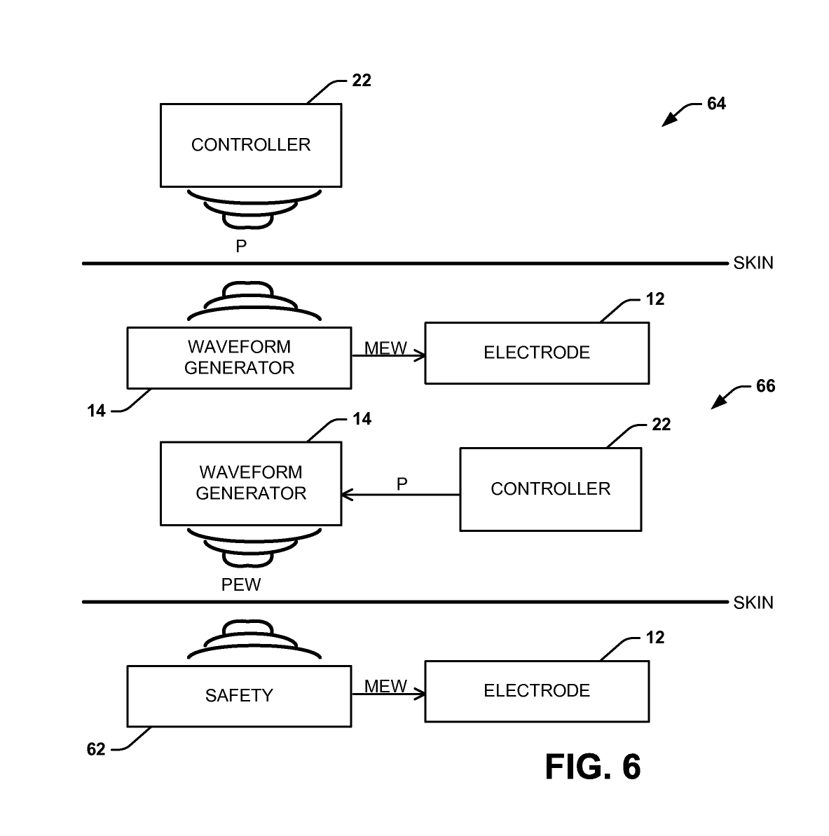

FIG. 6 shows example illustrations 64, 66 of how the system 20 can be configured both internally and externally to the patient's body. Illustration 64 shows the controller 22 external to the body and the waveform generator 14 and the electrode 12 internal to the body. The controller 22 can wirelessly transmit the altered parameter (P) to the waveform generator 14. The waveform generator 14 can alter the electrical signal according to the parameter (P) and provide the altered electrical signal (MEW) to the electrode 12, which can deliver the incomplete, bi-directional block corresponding to the altered electrical signal (MEW) to the nerve.

Illustration 66 shows the controller 22 and a portion of the waveform generator 14 external to the patient's body and the electrode 12 and a second portion of the waveform generator (e.g., a safety check) 62 internal to the patient. The controller 22 can wirelessly transmit the altered parameter (P) to the waveform generator 14. The waveform generator 14 can change the electrical signal according to the altered parameter and wirelessly transmit the proposed electrical signal (PEW) to the internal second portion of the waveform generator 62, which can perform a safety check (e.g., determining whether the proposed electrical signal (PEW) falls beyond a safety boundary and/or an efficacy boundary. If the proposed electrical signal (PEW) is beyond the safety boundary or the efficacy boundary, the second portion of the waveform generator 62 rejects the proposed electrical signal (PEW). If the proposed electrical signal (PEW) is within the safety boundary and the efficacy boundary, the second portion of the waveform generator 62 accepts the proposed electrical signal (PEW) as a modified electrical signal (MEW). The second portion of the waveform generator 62 can provide the modified electrical signal (MEW) to the electrode 12, which can generate and/or provide the incomplete, bi-directional block corresponding to the modified electrical signal to the nerve.

### IV. Methods

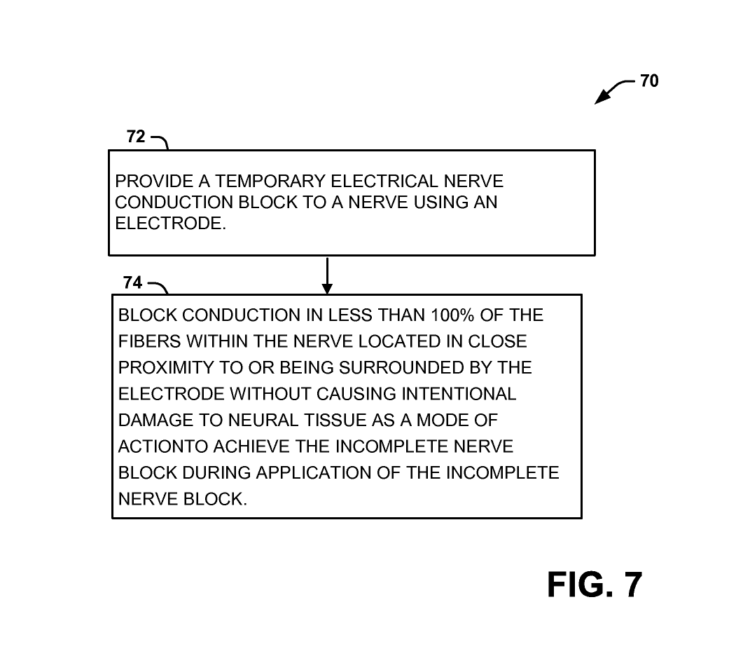

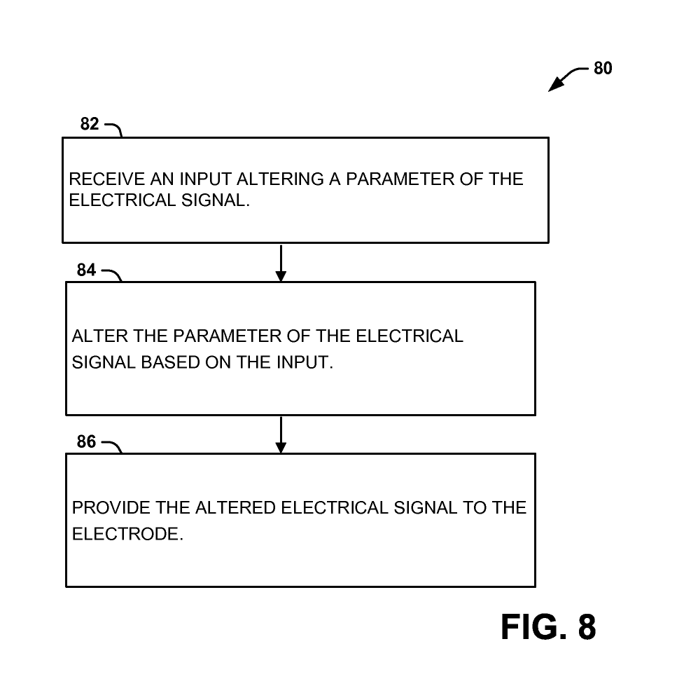

Another aspect of the present disclosure can include methods that can provide a temporary, incomplete, bi-directional nerve block to a patient, according to an aspect of the present disclosure. An example of a method 70 that can provide an incomplete, bi-directional electrical nerve block to a patient is shown in FIG. 7. Another example of a method 80 that can alter a parameter of the incomplete electrical nerve block (e.g., applied via the method 70) is shown in FIG. 8.

The methods 70 and 80 of FIGS. 7 and 8, respectively, are illustrated as process flow diagrams with flowchart illustrations. For purposes of simplicity, the methods 70 and 80 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 70 and 80.

Referring to FIG. 7, an aspect of the present disclosure can include a method 70 for providing an incomplete, bi-directional electrical nerve block to a patient. Incomplete electrical nerve conduction block can block conduction in less than 100% of the fibers within a nerve. The incomplete electrical nerve conduction block can be temporary. Advantageously, the incomplete electrical nerve conduction block can be applied to the portion of the nerve without intentionally damaging the nerve during application of the electrical nerve conduction block.

At 72, a temporary electrical nerve conduction block (e.g., EW) can be provided to a nerve using an electrode (e.g., electrode 12 with one or more contacts). For example, a waveform generator 14 can provide the signal to the electrode 12 for application to the nerve. In some instances, one or more parameters of the signal can be altered based on an input (e.g., from the patient and/or based on a status of the patient's body from a sensor). In some instances, the change of the one or more parameters can relate to the intensity of the incomplete nerve block. For example, the intensity of the block can be changed between values from 0% to less than 100% so that the block can be a graded nerve block.

At 74, conduction can be blocked bi-directionally in less than 100% of the fibers within the nerve located in close proximity to or being surrounded by the electrode. In some instances, the incomplete nerve block can be temporary, with a finite duration as defined by the temporary electrical nerve conduction block signal. Advantageously, the conduction can be blocked without causing intentional damage to neural tissue as a mode of action to achieve the incomplete nerve block during application of the incomplete nerve block.

The incomplete electrical nerve block can be used for many different applications. In some instances, the incomplete electrical nerve block can be used for spasticity control (e.g., providing variable levels of block for different patient activity levels). In other instances, the incomplete electrical nerve block can block fibers contributing to an unwanted action, while allowing conduction through fibers that do not contribute to the unwanted action (e.g., blocking motor fibers, while allowing conduction through sensory fibers). In still other instances, the incomplete nerve block can drive small fibers within a nerve, while blocking large fibers within the nerve (e.g., for select muscle drive of slow-fatiguing muscles). In further instances, the incomplete nerve block can permit selection between sympathetic and parasympathetic fibers (e.g., within nerves spanning between organs and the central nervous system). In still further instances, the incomplete electrical nerve block can be used as a filter combined with full nerve drive to achieve small fiber activation without activating the large fibers within the nerve. In yet other instances, the incomplete nerve block can be applied by one contact of an electrode (e.g., a nerve re-shaping electrode, such as a flat interface nerve electrode) and no block or a driving electrical stimulation can be applied by a second contact of the electrode. In still further instances, the incomplete nerve block can be a higher grade (intensity) block at night than during the day so that the patient can sleep at night, while having control and strength during the day to be able to walk (accepting some spasticity and pain in exchange for more strength and control).

Referring now to FIG. 8, another aspect of the present disclosure can include a method 80 for altering a parameter of the incomplete electrical nerve block. The method 80 of FIG. 8 disclosure may be embodied in hardware and/or in software (including firmware, resident software, micro-code, etc.). Furthermore, aspects of the present disclosure may take the form of a computer program product on a computer-usable or computer-readable storage medium having computer-usable or computer-readable program code embodied in the medium for use by or in connection with an instruction execution system. A computer-usable or computer-readable medium may be any non-transitory medium that can contain or store the program for use by or in connection with the instruction or execution of a system, apparatus, or device.

One or more blocks of the respective flowchart illustration of FIG. 8, and combinations of blocks in the block flowchart illustrations, can be implemented by computer program instructions. These computer program instructions can be stored in memory and provided to a processor of a general purpose computer, special purpose computer, and/or other programmable data processing apparatus to produce a machine, such that the instructions, which execute via the processor of the computer and/or other programmable data processing apparatus, create mechanisms for implementing the steps/acts specified in the flowchart blocks and/or the associated description. In other words, the steps/acts can be implemented by a system comprising a processor that can access the computer-executable instructions that are stored in a non-transitory memory. In some instances, the method 80 can be implemented by controller 22.

As illustrated in FIG. 8, the parameter of the electrical stimulus (e.g., EW) can be altered and the altered stimulus can be applied according to method 70 of FIG. 7. For example, the altered electrical nerve conduction block signal can be provided to the nerve via the electrode. Like the unaltered signal, the altered signal can provide a temporary nerve block with a different intensity. The altered signal can block the conduction in less than 100% of the fibers or sub-group of certain (diameter, functional, directional) fibers within the nerve (the same percentage of fibers as the signal or a different number of fibers from the signal) without intentionally damaging the nerve during application of the electrical nerve conduction block.

At 82, an input can be received (e.g., from a patient and/or from a sensor and related to a condition of the patient's body). The input can alter a parameter of the signal. In some instances, the input can be related to at least one of a plurality of predefined alterations of the parameter (e.g., stored in non-transitory memory 26 of the controller 22). In other instances, the input can be defined by the patient. In this case, the method can include the steps of checking whether the input alters the signal outside of a predefined safety boundary (e.g., defining whether the signal would injure the nerve) or a predefined efficacy boundary (e.g., defining whether the signal), and accepting or rejecting the input based on the predefined safety boundary and the predefined efficacy boundary. In further instances, the input can be received from a sensor and related to a condition of the patient's body.

At 84, the parameter of the signal can be altered based on the altered parameter. For example, the signal with the altered parameter can be the altered signal. At 86, the altered signal can be provided to the electrode for application to the nerve.

From the above description, those skilled in the art will perceive improvements, changes and modifications. Such improvements, changes and modifications are within the skill of one in the art and are intended to be covered by the appended claims.

What is claimed is:

1. A method for providing a bi-directional, incomplete nerve block to a patient, comprising the steps of:
providing an adjustable temporary electrical nerve conduction block to a nerve using an electrode,
wherein the temporary electrical nerve block is adjusted on demand and on the spot; and
blocking conduction in less than 100% of the fibers within the nerve located in close proximity to or being surrounded by the electrode without causing intentional damage of neural tissue as mode of action to achieve the incomplete nerve block during application of the electrical nerve conduction block,
wherein a complete recovery of nerve conduction is expected within seconds post application of the incomplete nerve block.

2. The method of claim 1, wherein the step of blocking the conduction further comprises at least one of:
blocking the conduction in large fibers within the nerve while permitting conduction in small fibers within the nerve;
blocking the conduction in small fibers within the nerve while permitting conduction in large fibers within the nerve;
blocking the conduction in efferent fibers within the nerve while permitting conduction in afferent fibers within the nerve;
blocking the conduction in afferent fibers within the nerve while permitting conduction in efferent fibers within the nerve;
blocking the conduction in sympathetic fibers within the nerve while permitting conduction in parasympathetic fibers within the nerve; and
blocking the conduction in parasympathetic fibers within the nerve while permitting conduction in sympathetic fibers within the nerve.

3. The method of claim 1, wherein the electrical signal comprises a high frequency electric alternating current (HFAC) waveform, a kilohertz HFAC (KHFAC) waveform, a charge-balanced direct current (CBDC) waveform, or a multi-phased direct current (MPDC) waveform.

4. The method of claim 1, wherein the step of blocking the conduction further comprises blocking the conduction in at least 10% and not more than 90% of the fibers within the nerve.

5. The method of claim 1, wherein the electrode comprises a plurality of contacts; and
wherein at least one of the plurality of contacts delivers the electrical signal to a portion of the nerve to incompletely block conduction within the portion of the nerve.

6. The method of claim 5, wherein a second one of the plurality of contacts delivers a second electrical signal to a second portion of the nerve to generate action potentials in fibers within the second portion of the nerve and
wherein the second portion of the nerve at least one of overlaps the first portion of the nerve and is separate from the first portion of the nerve.

7. The method of claim 1, further comprising adjusting a parameter of the electrical signal to block conduction within a different number of fibers within the nerve.

8. The method of claim 1, wherein the step of providing the adjustable temporary electrical nerve conduction block to the nerve further comprises the steps of:
receiving, by a system comprising a processor, an input altering a parameter of an electrical signal corresponding to the electrical nerve conduction block;
altering, by the system, the parameter of the electrical signal; and
providing, by the system, the altered electrical signal to the electrode for the electrical nerve conduction block.

9. The method of claim 8, wherein the input is related to at least one of a plurality of predefined alterations of the parameter of the electrical signal.

10. The method of claim 8, further comprising the steps of:
checking, by the system, whether the input alters the electrical waveform outside of a predefined safety boundary or a predefined efficacy boundary; and
accepting the input when the input alters the electrical waveform within the predefined safety boundary and the predefined efficacy boundary; or
rejecting the input when the input alters the electrical waveform outside of the predefined safety boundary or the predefined efficacy boundary.

11. A system that provides a bi-directional, incomplete nerve block to a patient, the system comprising:
a waveform generator configured to modulate a parameter of an electrical waveform so that the electrical waveform is configured to temporarily block conduction in less than 100% of the fibers within a portion of a nerve in close proximity to the electrode; and
an electrode, electrically coupled to the waveform generator, located in proximity to the nerve and configured to deliver the electrical conduction block waveform to the nerve via at least one contact.

12. The system of claim 11, further comprising:
a control unit communicatively coupled to the waveform generator and being configured to receive an input that modulates the parameter of the electrical waveform;
wherein the waveform generator is configured to modulate the parameter of the electrical waveform based on the input.

13. The system of claim 12, wherein at least one of an amplitude, a frequency, a polarity, a time period, and a shape of the waveform is adjusted based on the input.

14. The system of claim 12, wherein the control unit is configured to reject the input when the input modulates the parameter of the electrical waveform outside of a predefined safety boundary or predefined efficacy boundary.

15. The system of claim 11, wherein the electrical waveform comprises at least one of a HFAC waveform, a KHFAC waveform, a CBDC waveform, and a MPDC waveform.

16. The system of claim 11, wherein the waveform generator is electrically coupled to the electrode via at least one of a wire and an indirect coupling comprising at least one of capacitive coupling or inductive coupling.

17. The system of claim 11, further comprising a feedback unit configured to detect at least one of a patient physiological parameter, a patient activity, a patient position, a patient acceleration, a time of day, and a relative position of the patient's body, and to provide an input to the waveform generator adjusting the waveform in response to the detection.

18. The system of claim 11, further comprising a signal receiver located within the patient and coupled to the waveform generator, the signal receiver configured to provide the electrical waveform to the electrode;
wherein the waveform generator is located external to the patient.

19. The system of claim 11, wherein the electrode is a nerve shaping electrode, an electrode array, a spiral electrode, a cuff electrode, a Huntington style electrode, a co-linear placed spinal cord stimulation (SCS) or deep brain stimulation (DBS) electrode, a disk electrode, an intra-muscular electrode, or an intra-fascicular electrode.

20. A neural prosthesis comprising:
an external control unit configured to receive an input from a patient, the input modulating a parameter of an electrical waveform; and
a waveform generator configured to adjust the electrical waveform based on the input and provide the adjusted electrical waveform to a nerve of a patient;
wherein the electrical waveform is configured to provide a temporary, bi-directional, incomplete nerve block to the nerve at the electrode site.

21. The neutral prosthesis of claim 20, wherein the external control unit comprises a wireless transmitter and the waveform generator includes a receiver configured to receive a first signal from the wireless transmitter or a second signal from a sensor within the body;
wherein the external control unit is configured to wirelessly communicate the input to the waveform generator.

22. The neural prosthesis of claim 20, wherein the external control unit includes at least one predefined setting for the incomplete nerve block;
wherein the at least one predefined setting is stored in a memory of the external controller.

23. The neural prosthesis of claim 20, wherein the external controller comprises an external magnet and the input is a swipe of the magnet over the implanted waveform generator.

24. The neural prosthesis of claim 20, wherein the external control unit is configured to produce the input based on an activity level of the patient determined from an electroencephalogram (EEG), an electromyogram (EMG), an electrocardiogram (ECG), a breathing change, a pO₂ change, a pCO₂ change, an accelerometer measurement, a blood sugar change, a temperature change of the patient, or a specific time during the day/night cycle.

25. The neural prosthesis of claim 20, wherein the external control unit provides power to the waveform generator;
wherein power is not stored within the waveform generator; and
wherein the waveform generator is implanted within the patient.
